(19) United States
(12) Patent Application Publication
(10) Pub. No.: US 2022/0120750 A1
(43) Pub. Date: Apr. 21, 2022

(54) EXTRACELLULAR VESICLE BIOMARKERS FOR ENDOMETRIAL CANCER

(71) Applicant: Tymora Analytical Operations, Inc., West Lafayette, IN (US)

(72) Inventors: Anton Ilyuk, West Lafayette, IN (US); Weiguo A. Tao, West Lafayette, IN (US)

(21) Appl. No.: 17/503,473

(22) Filed: Oct. 18, 2021

Related U.S. Application Data
(60) Provisional application No. 63/093,152, filed on Oct. 16, 2020.

(51) Int. Cl. G01N 33/574

(52) U.S. Cl. CPC G01N 33/57442; G01N 33/57484

(57) ABSTRACT

Methods and products for the identification and detection of new endometrial cancer biomarkers based on proteins in plasma and/or uterine lavage extracellular vesicles.

### CROSS-REFERENCE TO RELATED APPLICATION

This U.S. patent application claims priority to U.S. Provisional Application No. 63/093,152 filed Oct. 16, 2020, to the above-named inventors, the disclosure of which is considered part of the disclosure of this application and is hereby incorporated by reference in its entirety.

### FEDERALLY SPONSORED RESEARCH OR DEVELOPMENT

Not applicable.

### SEQUENCE LISTING, A TABLE, OR A COMPUTER PROGRAM

Not applicable.

### FIELD OF THE INVENTION

This invention relates generally to a method to isolate proteins from biofluids, such as plasma or uterine lavage, for biomarker discovery or for clinical detection. More particularly, this invention relates to non-invasive early disease diagnosis, disease monitoring and disease classification. In one aspect, this invention relates to unique proteins capable of differentiating endometrial cancer plasma or uterine lavage samples from healthy samples and non-cancer conditions control samples. In another aspect, this invention relates to early-stage detection of endometrial cancer by plasma or uterine lavage test.

### BACKGROUND

Endometrial cancer (EC) is the most common gynecologic malignancy and the fourth most common cancer in women in the United States (1). Unlike most cancers, the incidence rates for endometrial cancer have been increasing, with the death rate going up by more than 100% over the past 20 years. By 2030, the projections show that endometrial cancer will surpass colorectal cancer and will become the third most common cancer in the U.S. women (2).

The primary reason behind this is the lack of effective screening methods that can detect pre-malignant lesions or early-stage cancers (3). When detected early, the survival rates for patients are very high (>95%) (4). On the other hand, the 5-year survival for the metastasized disease is <20%.

The most common symptom for women with endometrial cancer is abnormal uterine and postmenopausal bleeding, affecting ˜1.4 million women annually in the U.S. (5), although less than 10% of these cases will result in the cancer diagnosis (6-8).

The current “gold standard” for evaluating endometrial pathology after presenting these symptoms is hysteroscopy, which requires an operating room, anesthesia of the patient, and resulting in discomfort and high cost.

There is a critical need for a new diagnostic test that would avoid these drawbacks and enable more effective non-invasive detection of endometrial cancer at the earliest stage, which would significantly improve disease prognosis and survival rates.

In one aspect, this disclosure is related to a robust method for the identification and detection of new biomarkers based on proteins—a true measure of dynamic activity and cellular signaling, for the purposes of disease diagnosis, prognosis, detection, monitoring, patient stratification, drug response analysis, therapy selection, or the like.

The proposed method introduces a novel platform technology to isolate proteins and from biofluids, such as plasma or uterine lavage, for biomarker discovery or for clinical detection.

In another aspect, this disclosure is related to a method that successfully demonstrates the feasibility of developing biofluid-derived EV proteins for cancer profiling. It has tremendous transformative potential for early cancer diagnosis, monitoring and classification based on actual activated pathways using plasma or uterine lavage as the source.

Further, once fully established, the method of the present disclosure can be implemented by scientists worldwide to analyze the direct signaling networks for a cancer of interest in a non-invasive manner.

Furthermore, once fully established, these new biomarkers can be employed either isolated or as part of a panel of biomarkers as a liquid biopsy in clinical scenarios: (1) as a surveillance test in high-risk patients, such as those with high-risk cystic diseases, hereditary risk of cancer, among others or (2) as a liquid biopsy for the longitudinal monitoring of treatment response in patients with already established cancer diagnosis.

In yet another aspect, this disclosure relates to a biomarker panel for detection and monitoring of endometrial cancer. The approach will enable a truly non-invasive test and the first example of using EV proteins for early endometrial cancer diagnostics, especially in liquid biopsy setting.

Still further, it is envisioned to further apply this innovative procedure to validate and fully develop pre-determined biomarkers panels.

### BRIEF SUMMARY OF THE INVENTION

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

The first objective of this disclosure is to develop a process for the identification and detection of new biomarkers based on proteins—a true measure of dynamic activity and cellular signaling. The proposed method introduces a novel platform technology to isolate extracellular vesicles (EV) proteins from biofluids such as plasma and uterine lavage for biomarker discovery and clinical detection.

The method for capture, enrichment or isolation of extracellular vesicles is selected from the group consisting of Extracellular Vesicles total recovery and purification (EVtrap), ultracentrifugation (UC), filtrations, antibody-based purification, size-exclusion approach, polymer precipitation and affinity capture.

This disclosure is the first such method to successfully demonstrate the feasibility of developing uterine lavage- and plasma-derived EV proteins for endometrial cancer detection and profiling. It has tremendous transformative potential for early cancer diagnosis, monitoring and classification based on actual activated pathways using plasma and lavage as the source. Once fully established, it can be implemented by scientists worldwide to analyze the direct signaling networks for their cancer of interest in a non-invasive manner. It is envisioned to further apply this innovative procedure to validate and fully develop the disclosed current biomarker panel in Table I for detection and monitoring of endometrial cancer. The approach will enable a truly non-invasive test in liquid biopsy setting.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 is the graphical illustration of LC-MS proteome and phosphoproteome analyses of ultracentrifugation (e.g., 100K UC) and EVtrap™ (e.g., a novel extracellular vesicle total recovery and purification approach) samples (e.g., blood and/or uterine lavage samples). The fold change in overall signal between 100K UC and EVtrap™ experiments was quantified and plotted for: 88 identified common exosome markers, all proteins identified (e.g., excluding known contaminants), all phosphoproteins identified, high-abundant free plasma proteins (e.g., which are usually treated as contaminants), and serum albumin.

FIG. 2A is the multi-scatter plot accompanied with the Pearson correlation coefficients. A single plasma sample was separated into 6 aliquots and processed with our EVtrap™-LCMS protocol as 6 technical replicates.

FIG. 2B is the distribution plot of log (based 2) abundances of each protein by coefficient of variation (%).

FIG. 2C is the distribution plot of proteins by coefficient of variation (%).

FIG. 3 is the volcano plot representation of upregulated and downregulated EV proteins in patient samples derived from lavage with regard to endometrial cancer.

FIG. 4 is the heatmap representation of upregulated and downregulated EV proteins in patient samples derived from lavage with regard to endometrial cancer.

FIG. 5 is the volcano plot representation of upregulated and downregulated EV proteins in patient samples derived from plasma with regard to endometrial cancer.

FIG. 6 is the heatmap representation of upregulated and downregulated EV proteins in patient samples derived from plasma with regard to endometrial cancer.

FIG. 7 the graphical illustration of the violin plots of the select statistically upregulated proteins in patient samples derived from uterine lavage with regard to endometrial cancer.

FIG. 8A is the list of top 15 biomarkers discovered in patient samples derived from uterine lavage with regard to endometrial cancer, ranked by their permutation feature importance.

FIG. 8B is the ROC curve analysis of the top 15 EV proteins discovered in patient samples derived from uterine lavage with regard to endometrial cancer.

FIG. 9 is the confusion matrix of training and test datasets from patient samples derived from uterine lavage with regards to endometrial cancer.

FIG. 10 is the schematic illustration of two examples of decision trees generated from the data of new biomarkers discovered in patient samples derived from uterine lavage with regard to endometrial cancer.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

Unless otherwise stated, a reference to a compound or component includes the compound or component by itself, as well as in combination with other compounds or components, such as mixtures of compounds.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

All publications, patents and patent applications cited herein, whether supra or infra, are hereby incorporated by reference in their entirety to the same extent as if each individual publication, patent or patent application was specifically and individually indicated to be incorporated by reference.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

Developed Extracellular Vesicles total recovery and purification (EVtrap™) technology.

It is to be understood that any method for capture, enrichment or isolation of extracellular vesicles may be utilized, such as Extracellular Vesicles total recovery and purification (EVtrap), ultracentrifugation (UC), filtrations, antibody-based purification, size-exclusion approach, polymer precipitation, affinity capture and combination thereof.

In the preferred embodiment of the present disclosure, Evtrap™ is the preferred method. Total EV and/or exosome capture and purification has been the focus of many recent studies, with particular consideration toward simple and effective protocol. Vast majority of the exosome analysis projects are based on the differential centrifugation. In order to make the detection of EV biomarkers possible in a high-throughput automatable environment, a much faster and more robust method is needed. The goal of replacing ultracentrifugation (UC) as the method of choice is an important one, although not yet achieved by other proposed technologies. Here, this disclosure uses a novel non-antibody affinity beads-based capture method for effective EV isolation, termed EVtrap™ (Extracellular Vesicles total recovery and purification). It enables purification of the complete EV profile based on the lipid bilayer structure of these vesicles and the unique combination of the hydrophilic and aromatic lipophilic groups on the synthesized beads. The introductory manuscript of this technology was published in September 2018 (9).

In this disclosure, the inventors demonstrated the feasibility and clinical ability of the newly discovered plasma and uterine lavage biomarkers to discriminate between cancer and non-cancer samples, for the purposes of endometrial cancer diagnosis, prognosis, detection, monitoring, patient stratification, drug response analysis, therapy selection, or the like. This disclosure discovered several hundred EV proteins that appear to change significantly in endometrial cancer plasma and uterine lavage EVs compared to healthy or non-cancer conditions plasma and lavage samples. This disclosure narrowed this list down to about 123 potential markers that demonstrate the most statistically significant differentiation between the cases. Most of them show at least 4-fold change on a highly consistent and reproducible level at p-value<0.01. Table I lists unique uterine lavage/plasma EV proteins capable of differentiating endometrial cancer samples from healthy and non-cancer conditions control samples.

TABLE I
Biomarkers (uterine lavage/plasma EV proteins) capable
of differentiating endometrial cancer samples from
healthy and non-cancer conditions control samples

| | Foldchange | |
|---|---|---|
| Accession | (cancer/normal) | Gene Name |
| Q8N2M8 | 25.49 | CLASRP |
| Q15052 | 15.65 | ARHGEF6 |
| Q6IC98 | 14.44 | GRAMD4 |
| O15427 | 13.81 | SLC16A3 |
| Q96G28 | 13.24 | CFAP36 |
| Q96MU7 | 11.89 | YTHDC1 |
| O15204 | 11.80 | ADAMDEC1 |
| Q9Y3A3 | 9.59 | MOB4 |
| Q8NBF6 | 8.64 | AVL9 |
| Q96ST2 | 8.45 | IWS1 |
| O43566 | 7.70 | RGS14 |
| Q8TD84 | 7.58 | DSCAML1 |
| Q96HQ2 | 7.53 | CDKN2A/IPNL |
| Q9NP91 | 7.52 | SLC6A20 |
| Q6DD87 | 7.31 | ZNF787 |
| Q16853 | 7.07 | AOC3 |
| Q16637 | 6.80 | SMN1 |
| O75554 | 6.75 | WBP4 |
| Q5VZ52 | 6.75 | MORN5 |
| P60002 | 6.73 | ELOF1 |
| Q8N5H3 | 6.70 | FAM89B |
| Q5T5Y3 | 6.67 | CAMSAP1 |
| P46020 | 6.14 | PHKA1 |
| Q969R2 | 5.56 | OSBP2 |
| O00629 | 5.51 | KPNA4 |
| P21730 | 5.46 | C5AR1 |
| Q9UL03 | 5.38 | INTS6 |
| Q8N9B5 | 5.34 | JMY |
| Q15061 | 5.32 | WDR43 |
| P78556 | 5.31 | CCL20 |
| O14960 | 5.30 | LECT2 |
| Q02930 | 5.26 | CREB5 |
| Q9Y483 | 5.21 | MTF2 |
| P10145 | 5.07 | CXCL8 |
| P08572 | 5.02 | COL4A2 |
| O76031 | 4.99 | CLPX |
| Q6NUQ4 | 4.97 | TMEM214 |
| P67936 | 4.83 | TPM4 |
| P52657 | 4.66 | GTF2A2 |
| Q92765 | 4.62 | FRZB |
| Q4LDE5 | 4.48 | SVEP1 |
| Q13033 | 4.28 | STRN3 |
| Q96B26 | 4.26 | EXOSC8 |
| Q9H3Z4 | 4.16 | DNAJC5 |
| Q4VC05 | 4.08 | BCL7A |
| Q9NV35 | 4.03 | NUDT15 |
| Q92572 | 3.96 | AP3S1 |
| A8MTT3 | −15.76 | CEBPZOS |
| Q6E0U4 | −15.43 | DMKN |
| Q07075 | −12.23 | ENPEP |
| Q15819 | −11.09 | UBE2V2 |
| P04839 | 17.75 | CYBB |
| Q6X9E4 | 17.64 | FBXW12 |
| Q02338 | 17.24 | BDH1 |
| Q96GG9 | 15.61 | DCUN1D1 |
| Q0VDD8 | 15.47 | DNAH14 |
| P30512 | 15.23 | HLA-A |
| P06729 | 14.59 | CD2 |
| P14406 | 12.89 | COX7A2 |
| Q6ZVT6 | 12.61 | CFAP20DC |
| O95157 | 12.29 | NXPH3 |
| Q15102 | 10.83 | PAFAH1B3 |
| O75335 | 10.54 | PPFIA4 |
| Q8N5W9 | 10.36 | RFLNB |
| O95394 | 9.71 | PGM3 |
| P49767 | 9.71 | VEGFC |
| Q6ZRS2 | 9.62 | SRCAP |
| O75683 | 8.58 | SURF6 |
| Q63ZY3 | 8.29 | KANK2 |
| P46783 | 8.17 | RPS10 |
| Q7L5Y9 | 8.00 | MAEA |
| P07949 | 7.72 | RET |
| P21580 | 7.62 | TNFAIP3 |
| P55209 | 7.42 | NAP1L1 |
| Q53FT3 | 7.18 | HIKESHI |
| P56211 | 7.03 | ARPP19 |
| Q9UPZ9 | 7.02 | CILK1 |
| Q86V97 | 6.94 | KBTBD6 |
| Q8WWY8 | 6.79 | LIPH |
| P55285 | 6.56 | CDH6 |
| Q4KWH8 | 6.41 | PLCH1 |
| Q9H3K6 | 6.37 | BOLA2 |
| Q14532 | 6.34 | KRT32 |
| P35749 | 6.27 | MYH11 |
| Q9NRR3 | 5.84 | CDC42SE2 |
| P19440 | 5.83 | GGT1 |
| Q9P2H5 | 5.74 | USP35 |
| Q8NI22 | 5.60 | MCFD2 |
| Q15717 | 5.58 | ELAVL1 |
| Q6N069 | 5.56 | NAA16 |
| P17936 | 5.53 | IGFBP3 |
| Q6ZMI0 | 5.46 | PPP1R21 |
| Q15233 | 5.33 | NONO |
| Q9BSH5 | 5.33 | HDHD3 |
| Q9H0W9 | 5.23 | C11orf54 |
| Q15181 | 5.15 | PPA1 |
| Q99627 | 4.81 | COPS8 |
| Q9H4E7 | 4.70 | DEF6 |
| Q96K37 | 4.69 | SLC35E1 |
| O43678 | 4.61 | NDUFA2 |
| Q16537 | 4.43 | PPP2R5E |
| Q9HA77 | 4.37 | CARS2 |
| Q9Y3C8 | 4.31 | UFC1 |
| Q13796 | 4.29 | SHOOM2 |
| O95155 | 4.11 | UBE4B |
| P51858 | 4.11 | HDGF |
| Q6IQ22 | 4.03 | RAB12 |
| O75489 | 4.01 | NDUFS3 |
| O15347 | 3.17 | HMGB3 |
| P08865 | 1.72 | RPSA |
| P07093 | 11.07 | SERPINE2 |
| O60506 | 2.19 | SYNCRIP |
| P21741 | 5.21 | MDK |
| P00338 | 1.80 | LDHA |
| P61247 | 1.91 | RPS3A |
| O15069 | 2.18 | NACA |
| O75367 | 3.26 | MACROH2A1 |
| Q00688 | 1.67 | FKBP3 |
| Q13185 | 2.37 | CBX3 |
| P13984 | 1.98 | GTF2F2 |
| O00401 | 2.42 | WASL |
| P28676 | 1.91 | GCA |
| Q9NQW7 | 1.46 | XPNPEP1 |

EXAMPLE 1—Defining the exosomic proteomic landscape of endometrial cancer from plasma and uterine lavage fluid.

With aim to develop non-invasive biosignatures for EC diagnosis, we have completed a study using extracellular vesicles (EVs) from patient uterine lavage and plasma samples. We identified a large number of proteins from EVs in lavage and plasma by high-resolution mass spectrometry.

Our approach to date is the first method to successfully demonstrate the feasibility of profiling lavage-derived EV proteins for EC detection.

The proposed method introduces a novel platform technology to efficiently isolate EVs from human uterine lavage samples for biomarker discovery and clinical detection. It has tremendous transformative potential for early disease diagnosis and monitoring based on actual activated pathways using lavage as the non-invasive source.

While plasma has been the focus of the majority of non-invasive diagnostic tests currently in development, there is strong support that a more targeted sample source—uterine lavage—would provide a much better representation of a gynecological disease.

It has been known for decades that endometrial cancer exfoliates cells and other components into the uterine cavity (10). The success of the Papanicolaou (Pap) test in detection of cervical cancer is a great example of this.

For endometrial cancer, the use of uterine lavage (where saline is introduced into the uterine cavity and aspirated back) can offer a much more specific sample for diagnosing gynecologic malignancies. There is already evidence that somatic mutations related to endometrial cancer can be detected in lavage (11, 12). Unfortunately, mutations by themselves were not accurate enough to predict the presence of an active early disease, with almost 50% of non-cancer cases also presenting multiple driver mutations (3).

We proposed to use our expertise in the EV proteomics field to establish a robust EV isolation platform to screen and validate EC protein biomarkers from lavage and plasma.

Profiling of cell-secreted extracellular vesicles (EVs) offers all the same attractive advantages of a traditional liquid biopsy but without the sampling limitation of CTCs and ctDNA. These generally include smaller size exosomes derived from multivesicular endosome-based secretions, and microvesicles (MVs) derived from the plasma membrane (13-15).

The EVs provide an effective and ubiquitous method for intercellular communication and removal of excess materials and are utilized by every cell type studied to date. As these are shed into every biological fluid and embody a good representation of their parent cell, analysis of the EV cargo has great potential for biomarker discovery and disease diagnosis (16).

Notably, researchers have also found many differentiating characteristics of the cancer cell-derived cargo, including gene mutations, active miRNA and proteins, which possess metastatic properties (17-21). Particularly promising are the findings that these EV-based disease markers can be identified well before the onset of symptoms or physiological detection of a tumor. This makes them favorable candidates for early-stage cancer and other disease detection.

In addition, EVs are membrane-covered nanoparticles, which protects the inside contents from external proteases and other enzymes (22-24). We reasoned that these features make EVs a promising source to advance proteins as disease markers.

While there are existing methods that offer EV capture or precipitation, they typically have low recovery and/or purity. We have recently introduced a magnetic beads-based method, termed EVtrap™, to efficiently isolate EVs from biofluids for downstream proteomics analysis (9, 25). This provides a feasible path to develop EV-based disease diagnostics.

The use of uterine lavage or plasma with better diagnostic markers, such as EV proteins, could enable a highly tissue-specific non-invasive diagnostic test that can be performed during a routine office visit without the pain, discomfort, time and cost associated with hysteroscopy or uterine sonography.

Such an approach would offer a more sensitive, simple and cost-effective way to screen for endometrial cancer in women with increased risk of the disease. It would result in significant improvements in morbidity and mortality, and help remove barriers to testing and equal care that have plagued minorities and women of lower socioeconomic status (26).

To obtain more clinically relevant diagnostic information, we carried out comprehensive proteome analysis of extracellular vesicles released into the uterine lavage and blood plasma.

The uterine lavage and plasma samples were previously collected from women undergoing hysteroscopy and curettage in the operating room under anesthesia as a part of their gynecologic malignancies testing.

The samples were then separated into control (other non-cancer malignancies) and diagnosed endometrial cancer groups. This cohort provided valuable cancer differentiation information, where control samples were not from “healthy” individuals, but from women diagnosed with non-cancer conditions, such as uterine fibroids, adenomyosis, polyps, and ovulatory dysfunction. This mimics a real-case scenario where patients undergoing such a test would present symptoms and be a part of the high-risk group, instead of just disease/no disease comparison.

An extracellular vesicle total recovery and purification approach (EVtrap™) for EV capture from multiple biofluids was used. See, for example, Iliuk, A. et al., J Proteome Res 19, 2563-2574 (2020) (25); Wu, X. et al., J Proteome Res 17, 3308-3316 (2018) (9), which are incorporated by reference herein.

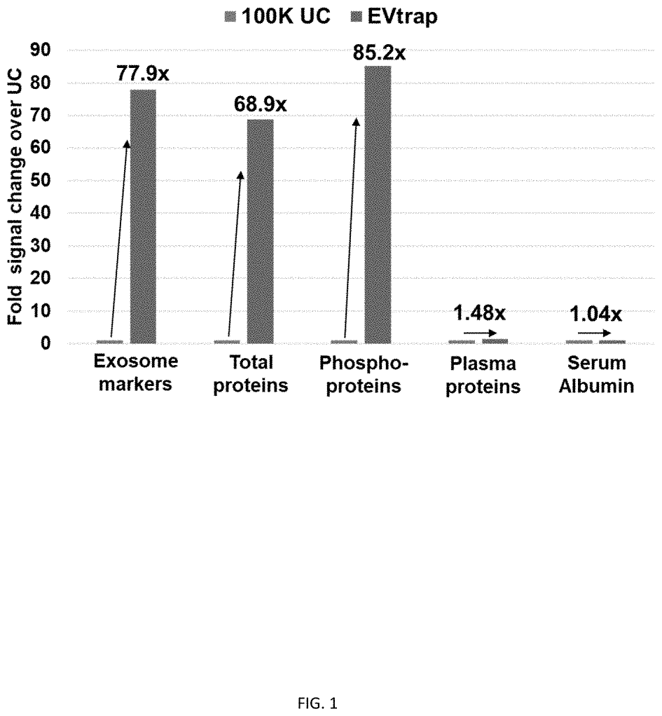

For our preliminary proteome analysis by EVtrap™ and liquid chromatography MS/MS (LC-MS/MS), 5 μL of plasma was used to identify >16,000 peptides from >2,200 unique proteins, with exceptionally superior results over an approach based on ultracentrifugation (FIG. 1).

Importantly, 95 out of 100 common ExoCarta-defined exosome markers were identified, all of which demonstrated a significant increase after EVtrap™ capture. This is noteworthy because many other studies have shown that different methods enrich different exosome populations with various success rates. With EVtrap™, it appears that the complete EV profile is recovered.

This same approach was applied with some modifications for this project to obtain complete proteomic profiles for purified plasma and uterine lavage EVs. EVs were isolated from plasma and uterine lavage samples using the EVtrap™ approach. EV proteins were extracted and trypsin digested with the aid of phase-transfer surfactants. After detergent removal and desalting, peptide samples were used for direct proteome analysis. Proteome fractions were analyzed on an Ultimate 3000 nanoLC apparatus coupled to a Q Exactive HF-X mass spectrometer.

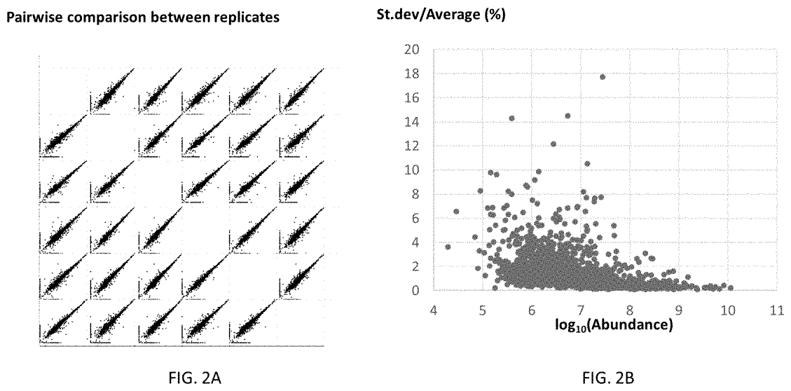

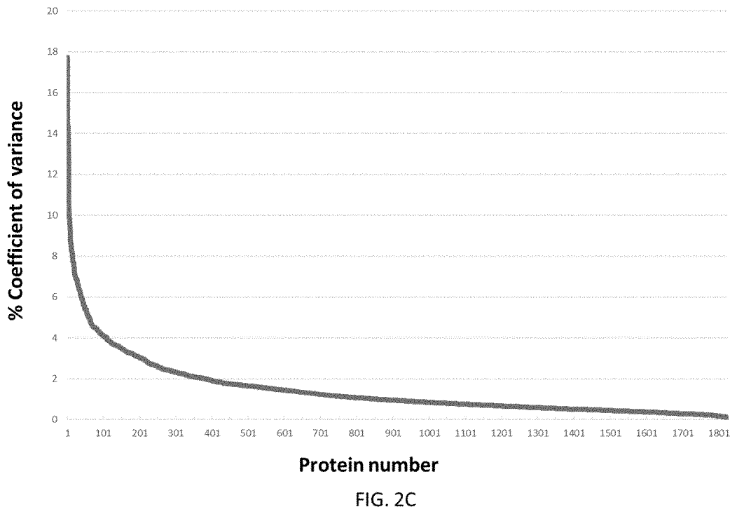

The reproducibility of protein abundances was assessed by measuring in six technical replicates of the same plasma or lavage sample from start to end of exosome isolation, purification, and protein identification pipeline (FIG. 2). Pairwise comparisons (FIG. 2A) demonstrate that the abundances of 1285 commonly assessed proteins are very well correlated (avg correlation coefficient=0.975). This replicate analysis revealed that the relative fluctuations in protein abundances or coefficient of variance (a ratio of standard deviation of abundance levels in replicates to average abundance level) decreases with increasing protein abundance levels (FIGS. 2B, 2C). Thus, the higher the protein abundance, the lower the coefficient of variance. The distribution of the absolute values for the coefficient of variance was also examined (FIG. 2C). The majority (53%) of protein markers displayed coefficient of variance <1%. Only a small fraction of markers (<2%) had relative fluctuations >5%. These data demonstrate excellent reproducibility of protein abundances using EVtrap™ followed by LC-MS analysis.

We then validated that our non-antibody affinity-based method, EVtrap™, was capable of enriching uterine lavage EVs at >95% recovery yield, >99% purity and with <5% coefficient of variation. These results mimic the data previously published for urine and plasma EVs (9, 25).

For the large-scale EV proteome biomarker discovery analysis, we utilized 132 uterine lavage and 132 plasma samples from patients diagnosed with endometrial cancer (62 samples) and non-cancer uterine conditions (70 samples). As an example, our lavage EV proteomic analyses resulted in the identification and quantitation of 46,240 peptide groups from 4,480 unique proteins.

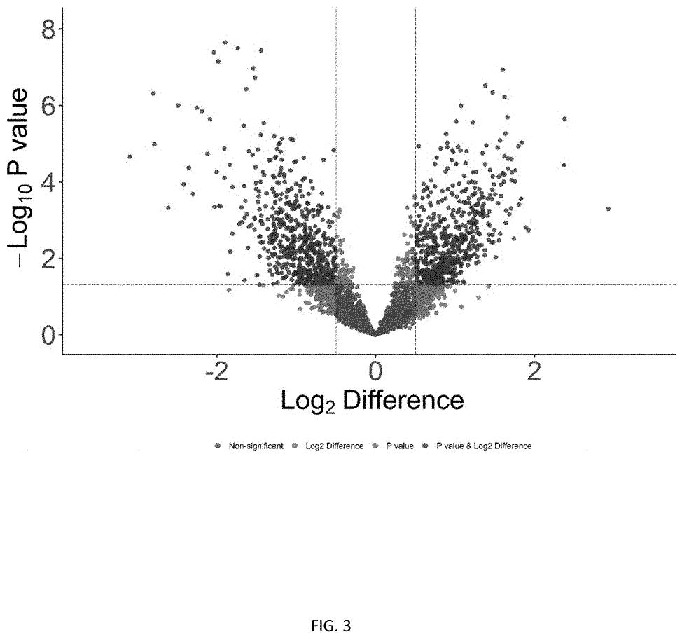

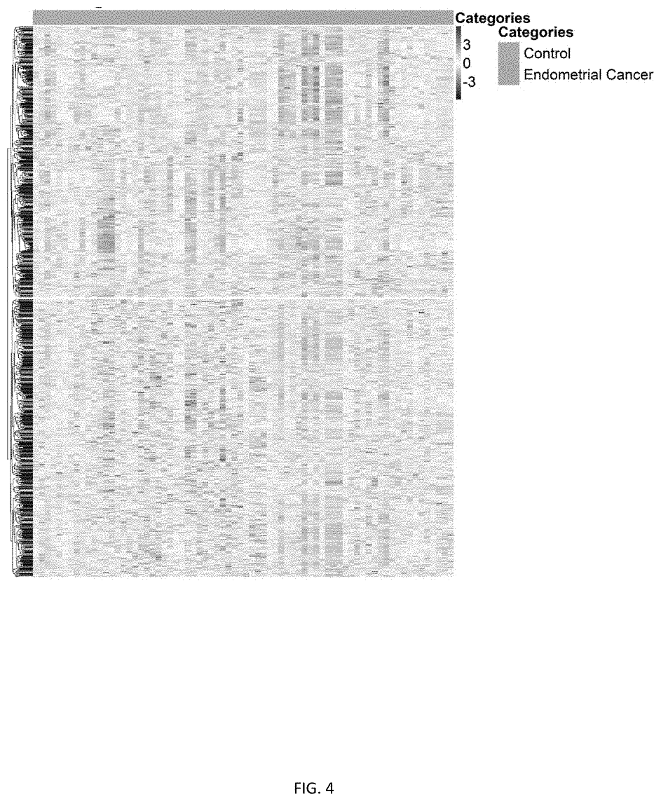

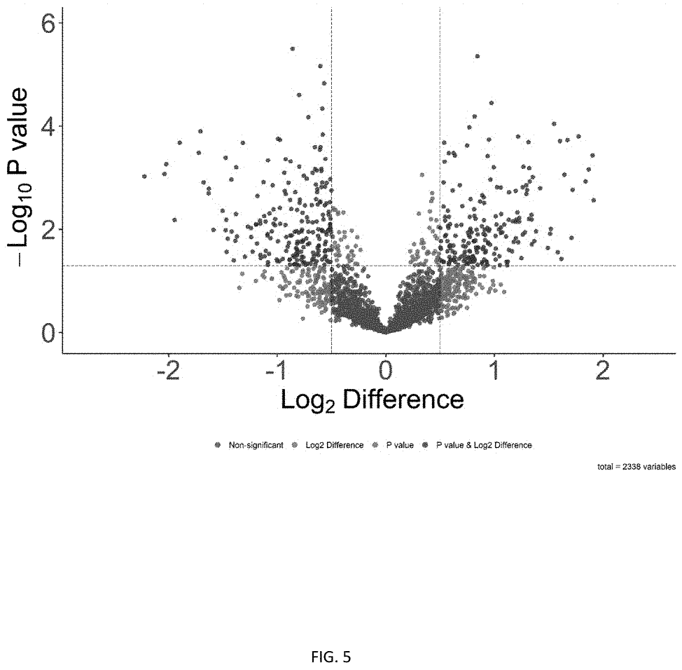

We identified several hundred upregulated/downregulated EV proteins that changed in abundance at statistically significant levels in endometrial cancer lavage samples at P-value<0.05. These lavage EV proteomics results are visualized in FIG. 3 with a volcano plot and FIG. 4 with a heatmap.

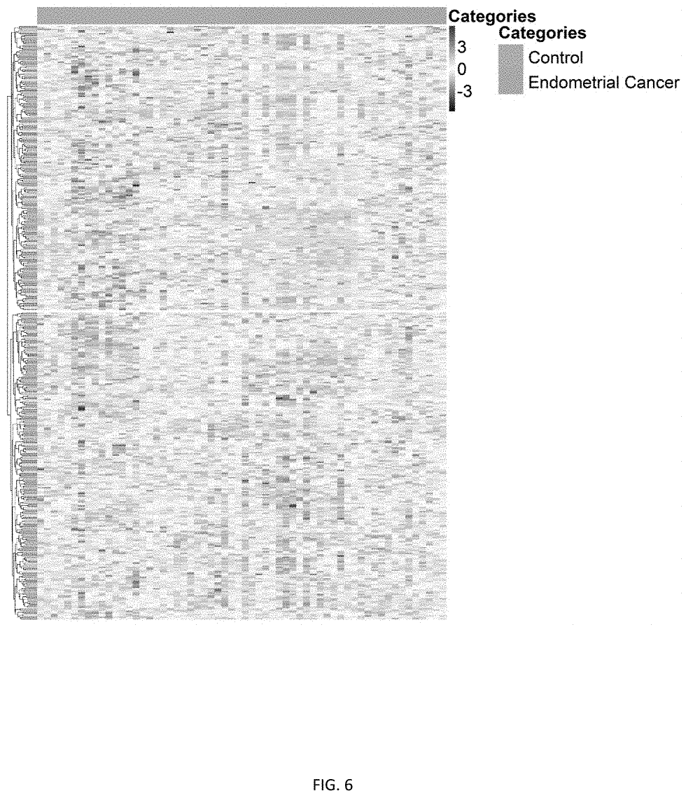

Likewise, we identified over one hundred upregulated/downregulated proteins that changed in abundance at statistically significant levels in endometrial cancer plasma samples at P-value<0.05. These plasma EV proteomics results are visualized in FIG. 5 with a volcano plot and FIG. 6 with a heatmap.

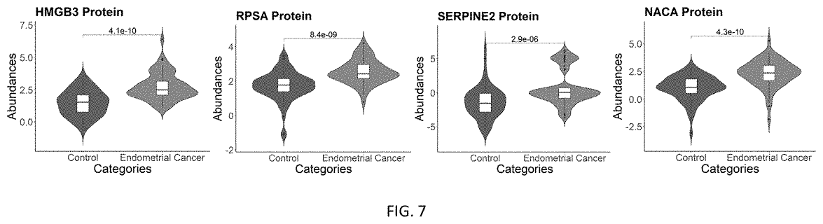

Examples of results for consistently differentiated lavage EV proteins are shown in the violin plots in FIG. 7, accompanied by the two-tail t-test P-values included on each of the violin plots.

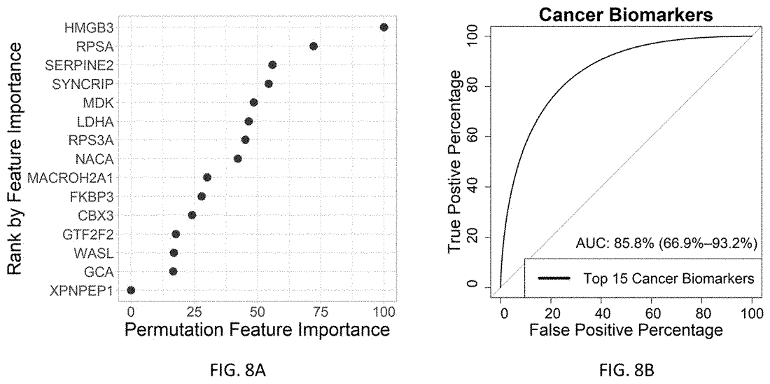

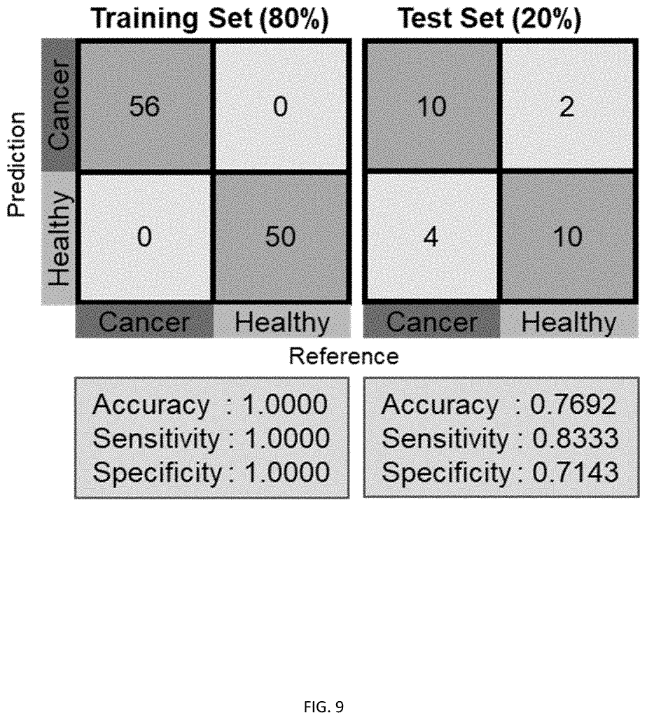

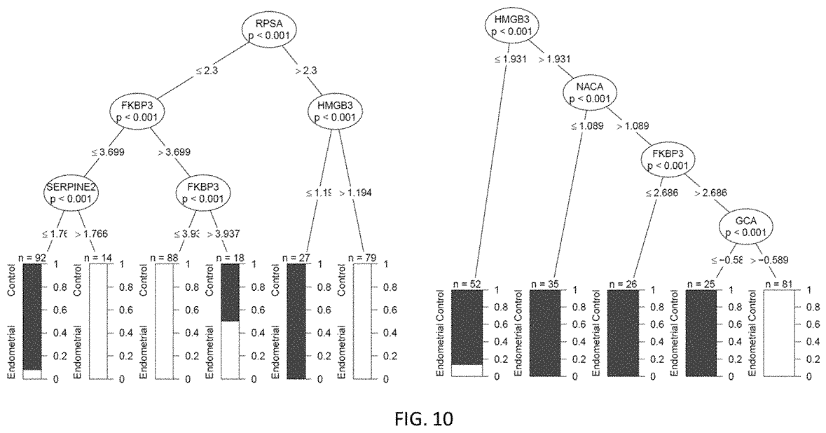

The samples from this dataset were subsequently split into a training set (80% of the samples) and test set (20%). The algorithm was trained using the training set, and then checked on the test set to see its accuracy. In the training set, a 10-fold cross validation was performed after selecting the top 15 features using the random forest (FIG. 8A). ROC curve was created for the top 15 features on the test set, which found that the best combination of markers can result in AUC of 85% (FIG. 8B). The confusion matrix of the training and test sets is illustrated in FIG. 9.

Our current statistical models are based on logistic regression and decision tree. These multivariable models allow simultaneous association of predictors and markers with clinical outcome. We carried out a preliminary analysis with our data and show two examples of decision trees generated in FIG. 10. An initial filter for biomarkers is set based on the fold change in intensity between the groups using Empirical Bayes linear models. Then the candidate proteins with high predictive capacity are selected by using the calculated importance in a random forest. Finally, we did the 10-fold cross-validation on the new data to train the models and predictive accuracy was assessed with the out-of-bag tests. Here, a cohort is split into 5 groups, and the model built on the first 4 (27). Then, the algorithm is used to predict the outcome of the last fifth subgroup. This process is repeated 10 times, each time rotating the test and training groups. Through such internal validation the significance of final findings is increased, reducing the probability of low-quality biomarkers and statistical overfitting. We utilized this model to compare the predictions with true outcomes and further refine our cut-off points and the algorithm, and reduce the number of markers in the panel.

Overall, these data confirm the ability of using the novel plasma or uterine lavage EV markers discovered as listed in Table I to successfully differentiate endometrial cancer samples from non-cancer controls.

Our biomarker discovery and validation results confirm the ability of using the novel lavage EV markers discovered by the method to successfully differentiate endometrial cancer samples from non-cancer controls. We believe that the AUC and diagnostic utility of this assay can be further improved using a larger sample cohort.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

### REFERENCES

1. Braun, M. M., Overbeek-Wager, E. A., and Grumbo, R. J. (2016) Diagnosis and Management of Endometrial Cancer. Am Fam Physician 93, 468-474.
2. Rahib, L., Smith, B. D., Aizenberg, R., Rosenzweig, A. B., Fleshman, J. M., and Matrisian, L. M. (2014) Projecting cancer incidence and deaths to 2030: the unexpected burden of thyroid, liver, and pancreas cancers in the United States. Cancer research 74, 2913-2921.
3. Nair, N., Camacho-Vanegas, O., Rykunov, D., Dashkoff, M., Camacho, S. C., Schumacher, C. A., Irish, J. C., Harkins, T. T., Freeman, E., Garcia, I., Pereira, E., Kendall, S., Belfer, R., Kalir, T., Sebra, R., Reva, B., Dottino, P., and Martignetti, J. A. (2016) Genomic Analysis of Uterine Lavage Fluid Detects Early Endometrial Cancers and Reveals a Prevalent Landscape of Driver Mutations in Women without Histopathologic Evidence of Cancer: A Prospective Cross-Sectional Study. PLoS Med 13, e1002206.
4. Siegel, R. L., Miller, K. D., and Jemal, A. (2016) Cancer statistics, 2016. CA Cancer J Clin 66, 7-30.
5. Matteson, K. A., Raker, C. A., Clark, M. A., and Frick, K. D. (2013) Abnormal uterine bleeding, health status, and usual source of medical care: analyses using the Medical Expenditures Panel Survey. J Womens Health (Larchmt) 22, 959-965.
6. Soleymani, E., Ziari, K., Rahmani, O., Dadpay, M., Taheri-Dolatabadi, M., Alizadeh, K., and Ghanbarzadeh, N. (2014) Histopathological findings of endometrial specimens in abnormal uterine bleeding. Arch Gynecol Obstet 289, 845-849.
7. Abid, M., Hashmi, A. A., Malik, B., Haroon, S., Faridi, N., Edhi, M. M., and Khan, M. (2014) Clinical pattern and spectrum of endometrial pathologies in patients with abnormal uterine bleeding in Pakistan: need to adopt a more conservative approach to treatment. Bmc Womens Health 14.
8. Clark, T. J., Voit, D., Gupta, J. K., Hyde, C., Song, F., and Khan, K. S. (2002) Accuracy of hysteroscopy in the diagnosis of endometrial cancer and hyperplasia: a systematic quantitative review. JAMA 288, 1610-1621.
9. Wu, X., Li, L., Iliuk, A., and Tao, W. A. (2018) Highly Efficient Phosphoproteome Capture and Analysis from Urinary Extracellular Vesicles. J Proteome Res 17, 3308-3316.
10. Traut, H. F., and Papanicolaou, G. N. (1943) Cancer of the Uterus: The Vaginal Smear in Its Diagnosis. Cal West Med 59, 121-122.
11. Maritschnegg, E., Wang, Y., Pecha, N., Horvat, R., Van Nieuwenhuysen, E., Vergote, I., Heitz, F., Sehouli, J., Kinde, I., Diaz, L. A., Jr., Papadopoulos, N., Kinzler, K. W., Vogelstein, B., Speiser, P., and Zeillinger, R. (2015) Lavage of the Uterine Cavity for Molecular Detection of Mullerian Duct Carcinomas: A Proof-of-Concept Study. Journal of clinical oncology: official journal of the American Society of Clinical Oncology 33, 4293-4300.
12. Martignetti, J. A., Pandya, D., Nagarsheth, N., Chen, Y., Camacho, O., Tomita, S., Brodman, M., Ascher-Walsh, C., Kolev, V., Cohen, S., Harkins, T. T., Schadt, E. E., Reva, B., Sebra, R., and Dottino, P. (2018) Detection of endometrial precancer by a targeted gynecologic cancer liquid biopsy. Cold Spring Harb Mol Case Stud 4.
13. Harel, M., Oren-Giladi, P., Kaidar-Person, O., Shaked, Y., and Geiger, T. (2015) Proteomics of microparticles with SILAC Quantification (PROMIS-Quan): a novel proteomic method for plasma biomarker quantification. Mol Cell Proteomics 14, 1127-1136.
14. Milane, L., Singh, A., Mattheolabakis, G., Suresh, M., and Amiji, M. M. (2015) Exosome mediated communication within the tumor microenvironment. J Control Release 219, 278-294.
15. Cocucci, E., and Meldolesi, J. (2015) Ectosomes and exosomes: shedding the confusion between extracellular vesicles. Trends in cell biology 25, 364-372.
16. Lin, J., Li, J., Huang, B., Liu, J., Chen, X., Chen, X. M., Xu, Y. M., Huang, L. F., and Wang, X. Z. (2015) Exosomes: novel biomarkers for clinical diagnosis. TheScientificWorldJournal 2015, 657086.
17. Melo, S. A., Luecke, L. B., Kahlert, C., Fernandez, A. F., Gammon, S. T., Kaye, J., LeBleu, V. S., Mittendorf, E. A., Weitz, J., Rahbari, N., Reissfelder, C., Pilarsky, C., Fraga, M. F., Piwnica-Worms, D., and Kalluri, R. (2015) Glypican-1 identifies cancer exosomes and detects early pancreatic cancer. Nature 523, 177-182.
18. Ridder, K., Sevko, A., Heide, J., Dams, M., Rupp, A. K., Macas, J., Starmann, J., Tjwa, M., Plate, K. H., Sultmann, H., Altevogt, P., Umansky, V., and Momma, S. (2015) Extracellular vesicle-mediated transfer of functional RNA in the tumor microenvironment. Oncoimmunology 4, e1008371.
19. Dobrowolski, R., and De Robertis, E. M. (2012) Endocytic control of growth factor signalling: multivesicular bodies as signalling organelles. Nat Rev Mol Cell Biol 13, 53-60.
20. Costa-Silva, B., Aiello, N. M., Ocean, A. J., Singh, S., Zhang, H., Thakur, B. K., Becker, A., Hoshino, A., Mark, M. T., Molina, H., Xiang, J., Zhang, T., Theilen, T. M., Garcia-Santos, G., Williams, C., Ararso, Y., Huang, Y., Rodrigues, G., Shen, T. L., Labori, K. J., Lothe, I. M., Kure, E. H., Hernandez, J., Doussot, A., Ebbesen, S. H., Grandgenett, P. M., Hollingsworth, M. A., Jain, M., Mallya, K., Batra, S. K., Jarnagin, W. R., Schwartz, R. E., Matei, I., Peinado, H., Stanger, B. Z., Bromberg, J., and Lyden, D. (2015) Pancreatic cancer exosomes initiate pre-metastatic niche formation in the liver. Nature cell biology 17, 816-826.
21. An, T., Qin, S., Xu, Y., Tang, Y., Huang, Y., Situ, B., Inal, J. M., and Zheng, L. (2015) Exosomes serve as tumour markers for personalized diagnostics owing to their important role in cancer metastasis. Journal of extracellular vesicles 4, 27522.
22. Sokolova, V., Ludwig, A. K., Hornung, S., Rotan, O., Horn, P. A., Epple, M., and Giebel, B. (2011) Characterisation of exosomes derived from human cells by nanoparticle tracking analysis and scanning electron microscopy. Colloids and surfaces. B, Biointerfaces 87, 146-150.
23. Boukouris, S., and Mathivanan, S. (2015) Exosomes in bodily fluids are a highly stable resource of disease biomarkers. Proteomics Clin Appl 9, 358-367.
24. van der Mijn, J. C., Sol, N., Mellema, W., Jimenez, C. R., Piersma, S. R., Dekker, H., Schutte, L. M., Smit, E. F., Broxterman, H. J., Skog, J., Tannous, B. A., Wurdinger, T., and Verheul, H. M. (2014) Analysis of AKT and ERK1/2 protein kinases in extracellular vesicles isolated from blood of patients with cancer. Journal of extracellular vesicles 3, 25657.
25. lliuk, A., Wu, X., Li, L., Sun, J., Hadisurya, M., Boris, R. S., and Tao, W. A. (2020) Plasma-Derived Extracellular Vesicle Phosphoproteomics through Chemical Affinity Purification. J Proteome Res 19, 2563-2574.
26. Collins, Y., Holcomb, K., Chapman-Davis, E., Khabele, D., and Farley, J. H. (2014) Gynecologic cancer disparities: a report from the Health Disparities Taskforce of the Society of Gynecologic Oncology. Gynecol Oncol 133, 353-361.
27. Shariat, S. F., Lotan, Y., Vickers, A., Karakiewicz, P. I., Schmitz-Drager, B. J., Goebell, P. J., and Malats, N. (2010) Statistical consideration for clinical biomarker research in bladder cancer. Urol Oncol 28, 389-400.

What is claimed is:

1. A compound comprising:
a biomarker for endometrial cancers consisting of plasma or uterine lavage extracellular vesicle (EV) proteins and any combination thereof, wherein each of the EV proteins or their combinations are capable of differentiating plasma or uterine lavage from a human with endometrial cancer from plasma or uterine lavage from a healthy human and plasma or uterine lavage from a human with non-cancer conditions, for the purposes of endometrial cancer diagnosis, prognosis, detection, monitoring, patient stratification, drug response analysis, therapy selection, or the like.

2. The compound of claim 1, wherein the biomarker has a putative compound identification, match form, name or pathway.

3. The compound of claim 1, wherein the biomarker is located on, in or about an extracellular vesicle.

4. The compound of claim 3, wherein the extracellular vesicle including the biomarker is captured, enriched or isolated using a method for capture, enrichment or isolation of extracellular vesicles.

5. The compound of claim 4, wherein the method for capture, enrichment or isolation of extracellular vesicles is selected from the group consisting of Extracellular Vesicles total recovery and purification (EVtrap), ultracentrifugation (UC), filtrations, antibody-based purification, size-exclusion approach, polymer precipitation and affinity capture.

6. The compound of claim 3, wherein the biomarker is detected from plasma or uterine lavage.

7. The compound of claim 6, wherein the biomarker is selected from a pre-determined biomarkers panel.

8. The compound of claim 3, wherein the extracellular vesicle is an exosome, microvesicle, endosome or other extracellular vesicle.

9. A method of detecting biomarkers comprising the steps of:
analyzing plasma or uterine lavage samples from humans with endometrial cancer, healthy controls, endometriosis, uterine fibroids, adenomyosis, polyps, ovulatory dysfunction, or other relevant conditions for an extracellular vesicle (EV) biomarker; and
detecting a biomarker in each plasma or uterine samples for the purposes of endometrial cancer diagnosis, prognosis, detection, monitoring, patient stratification, drug response analysis, therapy selection, or the like, wherein the biomarker consists of plasma or uterine lavage EV proteins and any combination thereof.

10. The method of claim 9, further comprising the step of:
analyzing differences in detected biomarkers between cancer and non-cancer plasma or uterine lavage samples, including observing that an EV proteomics of humans having endometrial cancer has clear separation from an EV proteomics of humans having non-cancer conditions or healthy controls.

11. The method of claim 10, further comprising the step of:
assessing the predictive capacity of detected biomarkers.

12. The method of claim 9, further comprising the step of:
identification of novel biomarkers.

13. The method of claim 9, wherein the biomarkers are selected from a pre-determined biomarkers panel.

14. A method of detecting biomarkers comprising the steps of:
isolating and capturing extracellular vesicles (EVs) from plasma or uterine lavage samples from humans endometrial cancer, healthy controls, endometriosis, uterine fibroids, adenomyosis, polyps, ovulatory dysfunction, or other relevant conditions for an extracellular vesicle (EV) biomarker, wherein the biomarker consists of plasma or uterine lavage EV proteins and any combination thereof;
analyzing the isolated and captured EVs by liquid chromatography-mass spectrometry, wherein the analysis step provides an EV protein profile (EV proteomics) for each plasma or uterine lavage sample; and
analyzing differences of the EV proteomics of humans having endometrial cancer and of the EV proteomics of humans having non-cancer conditions or healthy controls.

15. The method of claim 14, further comprising the step of:
processing and enrichment of the isolated and captured EVs prior to the liquid chromatography-mass spectrometry, filtering out soluble proteins and retaining EV associated proteins.

16. The method of claim 14, further comprising the step of:
performing biostatistical analysis in detected biomarkers between cancer and non-cancer controls including observing that the EV proteomics of humans having endometrial cancer has clear separation from the EV proteomics of humans having non-cancer conditions or healthy controls.

17. The method of claim 16, further comprising the step of:
assessing a disease predictive capacity of detected biomarkers.

18. The method of claim 17, further comprising the step of:
Identification of novel biomarkers.

19. The method of claim 14, wherein the biomarkers are selected from a pre-determined biomarkers panel.
